(19) United States
(12) Patent Application Publication
(10) Pub. No.: US 2013/0260458 A1
(43) Pub. Date: Oct. 3, 2013

(54) METHOD OF INDUCING DIFFERENTIATION OF HUMAN PLURIPOTENT STEM CELL INTO HEPATIC PROGENITOR CELL

(71) Applicant: NATIONAL UNIVERSITY CORPORATION CHIBA UNIVERSITY, Chiba-shi (JP)

(72) Inventor: Minoru TOMIZAWA, Chiba-shi (JP)

(73) Assignee: National University Corporation Chiba University, Chiba-shi (JP)

(21) Appl. No.: 13/852,627

(22) Filed: Mar. 28, 2013

(30) Foreign Application Priority Data
Mar. 30, 2012 (JP) 2012-080708
Mar. 21, 2013 (JP) 2013-058148

(51) Int. Cl. C12N 5/071

(52) U.S. Cl. CPC C12N 5/067
USPC 435/370; 435/366

(57) ABSTRACT

The present invention relates to a differentiation inducing method comprising: introducing, into human induced pluripotent stem cells, a combination of genes of transcription factors which are possessed by fetal and adult hepatic cells, but are not possessed by human induced pluripotent stem cells; and adhering the human induced pluripotent stem cells to a substrate for monolayer culture in a medium containing a combination of growth-promoting agents for proliferation and differentiation, thereby enabling induction of the differentiation of the human induced pluripotent stem cells in large amounts in a short term, a hepatic progenitor cell produced by the method, and a cell composition for transplantation into the liver comprising the cell. The transcription factors include FOXA2, GATA4, HEX and C/EBPα, and the growth-promoting agents include oncostatin M, an epidermal growth factor, retinoic acid, dexamethasone, insulin, transferrin and a selenite ion.

### CROSS-REFERENCE TO RELATED APPLICATIONS

This application claims priority to Japanese Patent Application Nos. 2012-080708, filed on Mar. 30, 2012, and 2013-058148, filed on Mar. 21, 2013, the entire contents of which are incorporated herein by reference.

### TECHNICAL FIELD

The present invention relates to a method of inducing the differentiation of a human induced pluripotent stem (iPS) cell into a hepatic progenitor cell, a hepatic progenitor cell induced by the method, and a cell composition for transplantation into the liver comprising the hepatic progenitor cell. More specifically, the present invention relates to a method of obtaining a hepatic progenitor cell by inducing the differentiation of an undifferentiated human pluripotent stem cell. Specifically, the invention relates to a method comprising a step of expressing a combination of gene products for differentiation induction in the human pluripotent stem cell which is adhered to a substrate for monolayer culture in a medium for differentiation. Further, the invention relates to a cell composition usable for transplantation into the liver which comprises the hepatic progenitor cell obtained by the method.

### BACKGROUND ART

The liver mainly includes hepatic cells which are hepatic parenchymal cells and hepatic non-parenchymal cells such as biliary epithelial cells. The liver is responsible for secretion of bile, filtration and detoxification of absorbed nutrients, drug metabolism, storage of sugar and control of blood glucose, and, additionally, is an organ which produces fibrinogen, heparin, anemia inhibitory substances and the like. Thus, the liver is essential for life. At present, the number of deaths due to hepatic cirrhosis exceeds 20,000 per year in Japan, and hepatic disease is the fifth leading cause of death. The number of chronic hepatitis cases is estimated to be about 1,300,000, and chronic active hepatitis accounts for approximately half thereof, which progresses to hepatic cirrhosis at about 3% per year.

On the other hand, techniques for differentiation of pluripotent stem cells such as embryonic stem cells (ES cells) and iPS cells into hepatic cells are expected to be applied to hepatic disease treatment, drug discovery and the like. For example, hepatic failure is a fatal pathological condition where functional hepatic cells are extremely reduced, and the transplantation of hepatic cells, if possible, can serve as a fundamental treatment strategy. Hepatic failure cases go into a very serious state such as bleeding tendency due to severe shortage of clotting factors or hepatic coma, and thus are demanded to be treated as soon as possible.

So, the transplantation of hepatic cells, if possible, can serve as a fundamental treatment strategy. It is expected to establish a method of inducing the differentiation of a stem cell into a hepatic cell in humans to transplant the differentiation-induced hepatic cell. Thus, there is an urgent need to develop a method of preparing a large number of hepatic cells from human iPS cells in a short term. For human pluripotent stem cells, however, a method of inducing the differentiation thereof into hepatic cells has not been established yet.

As for mice, a method of inducing the differentiation of ES cells (embryonic stem cells, abbreviated as “ES”) into hepatic cells (Non-Patent Document 2) and a method of separating such cells from other cells included together (Non-Patent Document 3) have also been developed. However, conventional methods for inducing the differentiation of embryonic stem cells into hepatic cells are based on the hanging drop method, and require formation of an embryoid body. Therefore, only a small amount of hepatic cells of interest are obtained by differentiation induction. For example, such methods are not suitable for supply of a large number of cells for dealing with fulminant hepatic failure, and it is impossible to produce a large number of hepatic cells. Further, conventional methods require three weeks, at minimum, to induce the differentiation of mouse ES cells into hepatic cells, and thus also cannot deal with emergent treatment. For example, in order to prepare a large number of hepatic cells, it is necessary to lay hepatic cells over the bottom face of a culture vessel for monolayer culture.

In addition, as for application to humans, there are present immunologic rejection and ethical problems because the use of human embryonic stem cells involves the use of embryonic stem cells derived from other people.

On the other hand, induced pluripotent stem cells (iPS cells) have been developed from human fibroblasts, and are expected to be applied to regenerative medicine (Non-Patent Document 4). However, clinical application of such cells to organ failure requires establishment of a method of inducing the differentiation thereof into cells of interest.

Therefore, the establishment of a method of induction from iPS cells which can be prepared from autologous cells and involve less problems such as immunologic rejection into hepatic cells is demanded. However, it is inferred that hepatic cells would undergo a complex process because they are differentiated while interacting with various cells in vivo, and the interaction with various cells cannot be reproduced by using cultured cells.

The differentiation of mouse ES cells into hepatic cells could be induced simply by forming embryoid bodies and culturing the embryoid bodies in a medium lacking glucose and arginine (Non-Patent Document 5). However, their undifferentiation potency is surprisingly maintained upon addition of activin, which causes formation of embryoid bodies in human iPS cells and induces differentiation into endoderm in mouse ES cells (Non-Patent Document 1). Accordingly, the method of preparing hepatic cells from mouse ES cells is not utterly efficient for attempts to produce hepatic cells from human iPS cells.

Further, there is an attempt to culture iPS cells in a culture dish in the presence of oncostatin M and retinoic acid to induce the differentiation thereof into hepatic cells (Non-Patent Document 6), but the differentiation inducing efficiency is insufficient to obtain a large number of differentiation-induced cells in a short term. This is because expression of GATA4, HEX and HNF3 (FoxA2), which are expressed in hepatic cells, is not observed. Therefore, this attempt is considered to result in insufficient differentiation induction of iPS cells into hepatic cells. The inventor therefore thinks that a large number of iPS cells cultured in a sheet form can be handled, but that it is impossible to induce the differentiation thereof into hepatic cells.

On the other hand, cells referred to as hepatic progenitor cells have been reported to have the abilities to proliferate actively and to differentiate into hepatic cells and biliary epithelium, which is found in fetal life (Non-Patent Document 7), and have small oval cells produced in the process of hepatic regeneration (Non-Patent Document 8), which have the proliferation capacity and the ability to differentiate into hepatic cells and biliary epithelial cells. Since hepatic progenitor cells have higher proliferation capacity than that of mature hepatic cells and form biliary epithelium as well, it is expected that, when transplanted into the liver, the hepatic progenitor cells can rapidly form the existing liver construction and efficiently reproduce an impaired liver as compared with the transplantation of hepatic cells alone (Non-Patent Document 8).

Recently, forced expression of HHEX (hereinafter referred to as “HEX”) genes in human ES cells and iPS cells has been reported to induce the differentiation of such cells into hepatic cells (Non-Patent Document 9). This method, however, requires replacement of a medium for passage culture with a first differentiation culture six days before the forced expression of HEX genes and further replacement of the first differentiation medium with a second differentiation medium one day before the forced expression. The expression of α-fetoprotein (AFP), as an indicator of differentiation induction, was found on the 15th day after transfer into the first differentiation medium, and the expression of albumin was found on the 18th day. Specifically, this reported method, even though carried out, would not be so different from the previously-described method of inducing the differentiation of mouse ES cells into hepatic cells in terms of the period required for differentiation induction.

Hepatic progenitor cells have both the ability to differentiate into hepatic cells and the ability to differentiate into biliary epithelium, and thus are expected to form hepatic cells and biliary epithelium when transplanted into the liver (Non-Patent Document 10). Thus, it is considered possible to efficiently reproduce an impaired liver as compared with the transplantation of hepatic cells alone.

Hepatic progenitor cells have both the ability to differentiate into hepatic cells and the ability to differentiate into biliary epithelium, and thus are expected to form hepatic cells and biliary epithelium when transplanted into the liver (Non-Patent Document 9). Thus, it is considered possible to efficiently reproduce an impaired liver as compared with the transplantation of hepatic cells alone.

### PRIOR-ART DOCUMENTS

### Non-Patent Documents

[Non-Patent Document 1] Tomizawa et al., Exp Therap Med 2: 405 (2011)
[Non-Patent Document 2] Yamamoto et al., Hepatology 37: 983 (2003)
[Non-Patent Document 3] Tomizawa et al., Cell Tissue RES 333: 17 (2008)
[Non-Patent Document 4] Takahashi et al., Cell 131: 861 (2007)
[Non-Patent Document 5] Zhon et al., J. Cell Biochem. 109: 606 (2010)
[Non-Patent Document 6] KANZO (Liver) 52 (Suppl 2): A680 (2011), published by the Japan Society of Hepatology, Tokyo, Japan
[Non-Patent Document 7] Kakinuma et al., J Hepatol 51: 127 (2009)
[Non-Patent Document 8] Sangan et al., Cell Tissue Res 342: 131 (2010)
[Non-Patent Document 9] Inamura et al., Molecular Therapy 19: 400 (2011)
[Non-Patent Document 10] Tomizawa et al., BiochemBiophys Res Commun 249: 1 (1998)

### SUMMARY OF THE INVENTION

There is an urgent need to develop a method of inducing the differentiation of human pluripotent stem cells and producing a large number of hepatic progenitor cells in a short term. However, a method which responds to this need, namely, a method of efficiently inducing the differentiation of human iPS cells into human hepatic progenitor cells has not been established yet.

The present inventor studied expression of genes associated with transcription factors which are observed to be expressed in fetal and adult hepatic cells, but are not observed to be expressed in human iPS cells, and determined the genes. The present inventor combined the transcription factor genes, introduced the combination of the transcription factors into human induced pluripotent stem cells, further studied a combination of growth-promoting agents for proliferation and differentiation of the gene-induced stem cells, and adhered the cells to a substrate for monolayer culture in a medium for differentiation, thereby establishing a method of inducing the differentiation of human iPS cells into hepatic progenitor cells. At last, the present invention has been completed.

The present invention relates to a method of inducing the differentiation of an undifferentiated human pluripotent stem cell to obtain a hepatic progenitor cell, comprising a step of expressing a combination of transcription factors for differentiation induction in the human pluripotent stem cell which is adhered to a substrate for monolayer culture in a medium for differentiation.

Further, the present invention relates to the method, wherein the transcription factors for differentiation induction are FOXA2, GATA4, HEX and C/EBPα.

Furthermore, the present invention relates to the method, wherein the medium for differentiation further comprises a combination of growth-promoting agents.

Also, the present invention relates to the method wherein the combination of growth-promoting agents includes one or more of the growth-promoting agents selected from the group consisting of oncostatin M, an epidermal growth factor, retinoic acid, dexamethasone, insulin, transferrin and a selenite ion.

Additionally, the present invention relates to the method wherein, in the step of expressing the combination of transcription factors for differentiation induction in the human pluripotent stem cell, the combination of transcription factors for differentiation induction is transiently expressed in the human pluripotent stem cell in a repetitive manner.

In addition, the present invention relates to any of the methods mentioned above, wherein the human pluripotent stem cell is a human induced pluripotent stem cell.

Also, the present invention relates to a cell composition for transplantation into the liver, comprising a human pluripotent stem cell-derived hepatic progenitor cell obtained by any of the methods mentioned above.

Furthermore, the present invention relates to a method of inducing the differentiation of human induced pluripotent stem cell (human iPS cell) to obtain a human hepatic progenitor cell, comprising steps of:

transfecting genes of transcription factors, FOXA2, GATA4, HEX and C/EBPα, respectively, into a human iPS cell every three days;

carrying out differentiation induction in a medium containing oncostatin M, an epidermal growth factor, retinoic acid, dexamethasone, insulin, transferrin and a selenite ion as a combination of growth-promoting agents; and,

obtaining a human hepatic progenitor cell by inducing differentiation of the human iPS cell, on the 8th day after the transfection.

Also, the present invention relates to a human hepatic progenitor cell obtained by inducing the differentiation of a human induced pluripotent stem cell (human iPS cell), wherein

genes of transcription factors, FOXA2, GATA4, HEX and C/EBPα, respectively, are transfected into a human iPS cell every three days, and

differentiation induction is carried out in a medium containing oncostatin M, an epidermal growth factor, retinoic acid, dexamethasone, insulin and transferrin as a combination of growth-promoting agents to obtain a human hepatic progenitor cell on the 8th day after the transfection.

Further, the present invention relates to a cell composition for transplantation into the liver, comprising the human hepatic progenitor cell.

Also, the present invention relates to a use of a system for expressing gene products of FOXA2, GATA4, HEX and C/EBPα in a human pluripotent stem cell, for the preparation of a human pluripotent stem cell-derived hepatic progenitor cell.

Further, the present invention relates to a use of a culture medium containing oncostatin M, an epidermal growth factor, retinoic acid, dexamethasone, insulin, transferrin and a selenite ion, for the preparation of a human pluripotent stem cell-derived hepatic progenitor cell.

Specifically, the present invention provides a method of inducing the differentiation of an undifferentiated human pluripotent stem cell to obtain a hepatic progenitor cell, comprising a step of expressing a combination of transcription factors for differentiation induction in the human iPS cell which is adhered to a substrate for monolayer culture in a medium for differentiation.

In the method, the combination of transcription factors for differentiation induction may include FOXA2, GATA4, HEX and C/EBPα.

In the method, the medium for differentiation may further comprise a medium containing a combination of growth-promoting agents.

In the method according to the present invention, the growth-promoting agents may be one or more of growth-promoting agents selected from the group consisting of oncostatin M, an epidermal growth factor, retinoic acid, dexamethasone, insulin, transferrin and a selenite ion.

Also, the present invention provides a method of inducing the differentiation of an undifferentiated human pluripotent stem cell to obtain a hepatic progenitor cell,

the method comprising a step of expressing a combination of transcription factors for differentiation induction in the human pluripotent stem cell which is adhered to a substrate for monolayer culture in a medium for differentiation,

wherein the medium for differentiation contains oncostatin M, an epidermal growth factor, retinoic acid, dexamethasone, insulin, transferrin and a selenite ion; and

the combination of transcription factors for differentiation induction includes FOXA2, GATA4, HEX and C/EBPα.

In the step of expressing the combination of transcription factors for differentiation induction in the human pluripotent stem cell according to the present invention, the combination of transcription factors for differentiation induction may be transiently expressed in the human pluripotent stem cell in a repetitive manner.

In the present invention, the human pluripotent stem cell may be a human induced pluripotent stem cell or an embryonic pluripotent stem cell.

The present invention may relate to a cell composition for transplantation into the liver, comprising the human pluripotent stem cell-derived hepatic progenitor cell obtained by this method.

Further, the present invention provides a method of inducing the differentiation of human induced pluripotent stem cell (human iPS cell) to obtain a human hepatic progenitor cell, comprising steps of:

transfecting genes of transcription factors, FOXA2, GATA4, HEX and C/EBPα, respectively, into a human iPS cell every three days;

further carrying out differentiation induction in a medium containing oncostatin M, an epidermal growth factor, retinoic acid, dexamethasone, insulin, transferrin and a selenite ion as a combination of growth-promoting agents; and

obtaining a human hepatic progenitor cell differentiation-induced from the human iPS cell, on the 8th day after the transfection.

Also, the present invention provides a human hepatic progenitor cell obtained by inducing the differentiation of a human induced pluripotent stem cell (human iPS cell), wherein

genes of transcription factors, FOXA2, GATA4, HEX and C/EBPα, respectively, are transfected into a human iPS cell every three days, and

differentiation induction is carried out in a medium containing oncostatin M, an epidermal growth factor, retinoic acid, dexamethasone, insulin, transferrin and a selenite ion as a combination of growth-promoting agents, whereby a human hepatic progenitor cell is obtained on the 8th day after the transfection.

The present invention provides a composition for transplantation into the liver, comprising the human hepatic progenitor cell.

The present invention also provides a use of a system for expressing gene products of FOXA2, GATA4, HEX and C/EBPα in a human pluripotent stem cell, for the preparation of a human pluripotent stem cell-derived hepatic progenitor cell.

The present invention also provides a use of a culture medium containing oncostatin M, an epidermal growth factor, retinoic acid, dexamethasone, insulin, transferrin and a selenite ion, for the preparation of a human pluripotent stem cell-derived hepatic progenitor cell.

Pluripotent stem cells are stem cells having the self-replication ability for a long term under predetermined culture conditions and having the pluripotency to differentiate into various cells under predetermined differentiation induction conditions, and can be produced by a known method (but not limited thereto) (Takahashi et al., Cell 131: 861 (2007)).

The induction of the differentiation of a pluripotent stem cell into a hepatic progenitor cell according to the present invention involves a step of introducing, into a pluripotent stem cell, genes of transcription factors which are observed to be expressed in fetal or adult hepatic cells, but are not observed to be expressed in pluripotent stem cells.

In the present invention, the term “transcription factors” refers to a group of proteins specifically binding to DNA. They bind to transcription regulatory regions, which are referred to as promoters and enhancers, on DNA to promote or conversely suppress the process of transcribing the genetic information on DNA to RNA. Transcription factors exert this function alone or in a complex with other proteins.

The transcription factors of which genes are expressed in fetal or adult hepatic cells, but are not expressed in pluripotent stem cells include FOXA2, GATA4, HEX and C/EBPα, but not limited thereto.

The term “FOXA2” as used herein refers to a human FORKHEAD box A2 gene. The amino acid sequence of FOXA2 protein is listed in SEQ ID NO: 27.

In the present invention, FOXA2 protein includes deletion, substitution or insertion of one or several amino acid(s) in the amino acid sequence listed in SEQ ID NO: 27, provided that the differentiation of undifferentiated human pluripotent stem cells is induced to obtain hepatic progenitor cells. Or, the FOXA2 protein has a 80% or more, 90% or more, or 95% or more identity to the amino acid sequence listed in SEQ ID NO: 27, provided that the differentiation of undifferentiated human pluripotent stem cells is induced to obtain hepatic progenitor cells. The polynucleotide of the FOXA2 is a nucleotide sequence including deletion, substitution or insertion of one or several nucleotide(s) in the nucleotide sequence coding the amino acid sequence listed in SEQ ID NO: 27, provided that the protein encoded by the polynucleotide induces the differentiation of undifferentiated human pluripotent stem cells to obtain hepatic progenitor cells. The polynucleotide of the FOXA2 is a nucleotide sequence which can be hybridized to the nucleotide sequence coding the amino acid sequence listed in SEQ ID NO: 27 under stringent conditions, provided that the protein encoded by the polynucleotide induces the differentiation of undifferentiated human pluripotent stem cells to obtain hepatic progenitor cells.

The term “GATA4” as used herein refers to a human GATA binding protein 4 gene. The amino acid sequence of GATA4 protein is listed in SEQ ID NO: 28.

In the present invention, GATA4 protein includes deletion, substitution or insertion of one or several amino acid(s) in the amino acid sequence listed in SEQ ID NO: 28, provided that the differentiation of undifferentiated human pluripotent stem cells is induced to obtain hepatic progenitor cells. Or, the GATA4 protein has a 80% or more, 90% or more, or 95% or more identity to the amino acid sequence listed in SEQ ID NO: 28, provided that the differentiation of undifferentiated human pluripotent stem cells is induced to obtain hepatic progenitor cells. The polynucleotide of the GATA4 is a nucleotide sequence including deletion, substitution or insertion of one or several nucleotide(s) in the nucleotide sequence coding the amino acid sequence listed in SEQ ID NO: 28, provided that the protein encoded by the polynucleotide induces the differentiation of undifferentiated human pluripotent stem cells to obtain hepatic progenitor cells. The polynucleotide of the GATA4 is a nucleotide sequence which can be hybridized to the nucleotide sequence coding the amino acid sequence listed in SEQ ID NO: 28 under stringent conditions, provided that the protein encoded by the polynucleotide induces the differentiation of undifferentiated human pluripotent stem cells to obtain hepatic progenitor cells.

The term “HEX” as used herein refers to an HHEX, namely, Hematopoietically Expressed Homeobox gene. The amino acid sequence of HEX protein is listed in SEQ ID NO: 29.

In the present invention, HEX protein includes deletion, substitution or insertion of one or several amino acid(s) in the amino acid sequence listed in SEQ ID NO: 29, provided that the differentiation of undifferentiated human pluripotent stem cells is induced to obtain hepatic progenitor cells. Or, the HEX protein has a 80% or more, 90% or more, or 95% or more identity to the amino acid sequence listed in SEQ ID NO: 29, provided that the differentiation of undifferentiated human pluripotent stem cells is induced to obtain hepatic progenitor cells. The polynucleotide of the HEX is a nucleotide sequence including deletion, substitution or insertion of one or several nucleotide(s) in the nucleotide sequence coding the amino acid sequence listed in SEQ ID NO: 29, provided that the protein encoded by the polynucleotide induces the differentiation of undifferentiated human pluripotent stem cells to obtain hepatic progenitor cells. The polynucleotide of the HEX is a nucleotide sequence which can be hybridized to the nucleotide sequence coding the amino acid sequence listed in SEQ ID NO: 29 under stringent conditions, provided that the protein encoded by the polynucleotide induces the differentiation of undifferentiated human pluripotent stem cells to obtain hepatic progenitor cells.

The term “CEBPA” as used herein refers to a human CCAAT enhancer binding protein alpha gene. The amino acid sequence of CEBPA protein is listed in SEQ ID NO: 30.

In the present invention, CEBPA protein includes deletion, substitution or insertion of one or several amino acid(s) in the amino acid sequence listed in SEQ ID NO: 30, provided that the differentiation of undifferentiated human pluripotent stem cells is induced to obtain hepatic progenitor cells. Or, the CEBPA protein has a 80% or more, 90% or more, or 95% or more identity to the amino acid sequence listed in SEQ ID NO: 30, provided that the differentiation of undifferentiated human pluripotent stem cells is induced to obtain hepatic progenitor cells. The polynucleotide of the CEBPA is a nucleotide sequence including deletion, substitution or insertion of one or several nucleotide(s) in the nucleotide sequence coding the amino acid sequence listed in SEQ ID NO: 30, provided that the protein encoded by the polynucleotide induces the differentiation of undifferentiated human pluripotent stem cells to obtain hepatic progenitor cells. The polynucleotide of the CEBPA is a nucleotide sequence which can be hybridized to the nucleotide sequence coding the amino acid sequence listed in SEQ ID NO: 30 under stringent conditions, provided that the protein encoded by the polynucleotide induces the differentiation of undifferentiated human pluripotent stem cells to obtain hepatic progenitor cells.

In the present invention, the homology of nucleotide sequences and amino acid sequences can be calculated by using the sequence alignment program CLUSTALW which is well known to those skilled in the art.

The term “stringent conditions” as used herein means that experiments are made under the following experimental conditions by the Southern blot method explained in Sambrook, J. and Russell, D. W., Molecular Cloning A Laboratory Manual 3rd Edition, Cold Spring Harbor Laboratory PrESs (2001). A polynucleotide comprising a nucleotide sequence to be compared is allowed to form a band by agarose electrophoresis, and then immobilized on a nitrocellulose filter or any other solid phase by a capillary phenomenon or electrophoresis. It is pre-washed with a solution containing 6×SSC and 0.2% SDS. A hybridization reaction between a probe in which a polynucleotide comprising the nucleotide sequence of the present invention is labeled with a radioisotope or any other labeling substance and the polynucleotide to be compared which has been immobilized on the solid phase is performed overnight at 65° C. in a solution containing 6×SSC and 0.2% SDS. Thereafter, the solid phase is washed twice at 65° C. in a solution containing 1×SSC and 0.1% SDS for 30 minutes each, and then washed twice at 65° C. in a solution containing 0.2×SSC and 0.1% SDS for 30 minutes each. Finally, the amount of the probe remaining on the solid phase is determined via quantitative determination of the labeling substance. The term “hybridized ‘under stringent conditions’” as used herein means that the amount of the probe remaining on the solid phase on which the polynucleotide comprising the nucleotide sequence to be compared has been immobilized is at least 25%, preferably at least 50%, more preferably at least 75% or more of the amount of the probe remaining on the solid phase in a positive control experiment, on which the polynucleotide comprising the nucleotide sequence of the present invention has been immobilized.

The phrase “system for expressing gene products of FOXA2, GATA4, HEX and CEBPA in human pluripotent stem cells” refers to any of systems for expressing the gene products in human pluripotent stem cells. The system may be an expression vector which expresses the gene products in a human pluripotent stem cell. The expression vector may be a plasmid, a virus or any other well-known vector. The expression vector preferably includes a gene expression regulatory sequence which expresses the gene products in a cell type of cell lineage from undifferentiated human pluripotent stem cells to hepatic progenitor cells, i.e., a promoter and/or an enhancer. The system may include a reagent for transfecting such an expression vector into a human pluripotent stem cell, for example, a reagent for lipofection, or an apparatus for electroporation. Or, a protein which is incorporated into a human pluripotent stem cell and can work therein, for example, a fusion protein coupled to a cell membrane permeable peptide may be included in the system. Such a protein is incorporated in a human pluripotent stem cell just upon addition to a medium, and thus is more preferred than the expression vector in that the protein does not permanently remain in the differentiated hepatic progenitor cell.

The phrase “cell membrane permeable peptide” as used herein refers to such a peptide which, upon extracellular addition of a fusion protein thereof with another peptide, polypeptide or protein, can permeate cell membrane to transfer the fusion protein into cells, and the peptide includes, but not limited to, an arginine-rich basic peptide (TAT peptide) derived from the Tat protein RNA binding region (positions 48 to 60) of human immunodeficiency virus type 1 (HIV-1), an homo-oligopeptide comprising 6 to 12 contiguous arginine residues, and a peptide (penetratin) having a basic amphipathic helix structure derived from the DNA binding region of a drosophila-derived transcription factor, Antennapedia protein.

In the present invention, the introduction of genes of transcription factors which are expressed in fetal or adult hepatic cells, but are not expressed in pluripotent stem cells into pluripotent stem cells can be realized by preparing an expression vector by a known method and transfecting the transcription factor genes into pluripotent stem cells by a method known in the art such as the lipofection or electropolation method.

In the present invention, endogenous protein which promotes the proliferation and differentiation of a specific cell in the mammalian animal body is referred to as a growth factor, and includes, but not limited to, oncostatin M and an epidermal growth factor, and, dexamethasone, insulin, transferrin and a selenite ion as growth factor aids having cell growth promoting effect. These growth factors and growth factor aids are collectively referred to as growth-promoting agents. In the present invention, human pluripotent stem cells were cultured in a medium containing the growth-promoting agents, resulting in successful induction of the differentiation of human pluripotent stem cells into hepatic progenitor cells.

In mouse ES cells, upon formation of embryoid bodies, a structure similar to endoderm in fetal life is formed (Abe et al., Exp Cell Res 229:27 (1996)). However, the cells influence with each other in a three-dimensional environment, and therefore are differentiated into various cells, resulting in the problem that cells other than the target cells are also present together. Thus, in the present invention, the induction of the differentiation of human pluripotent stem cells into hepatic progenitor cells was carried out through monolayer formation of cells adhered to a substrate without (while avoiding) formation of embryoid bodies of the cell.

A method involving adhering cells to a substrate for monolayer culture enables handling of a large number of cells in the same environment by simpler operations. Further, the experimental system is two-dimensional and simple, thereby making it possible to carryout the addition of growth-promoting agents and the introduction of transcription factor genes in an easy way and also making it possible to obtain a large number of target cells with less inclusion of other cells.

All the other differentiation inducing methods require a plurality of steps, whereas the method according to the present invention requires only a single step. The application of such a single step to the method of induction from iPS cells in the method of the present invention is novel.

The induction of the differentiation of pluripotent stem cells into hepatic progenitor cells can be confirmed by using, as indicators, increased production of α-fetoprotein (AFP) which is a marker for undifferentiated hepatic cells, increased expression of Delta like-1 (DLK-1) which controls the proliferation and differentiation of immature cells such as stem cells and progenitor cells and suggests the differentiation into hepatic cells, and/or increased expression of γ-GTP which is a marker for biliary epithelium, as well as incorporation of indocyanine green (ICG) reflecting drug metabolism as hepatic cells, in differentiation-induced cells (Tomizawa et al., BiochemBiophys Res Commun 249: 1 (1998), Inamura et al., Molecular Therapy 19: 400 (2011)). However, the indicators are not limited to these.

DLK-1 is observed to be expressed in hepatic cells in the fetal liver. The expression of DLK-1 disappears in the adult liver (Tanimizu et al., J Cell Sci., 116: 1775-1786, 2003), and it is used as a marker for hepatic progenitor cells (Tanimizu et al., Gene Expre. Patterns 5: 209-218, 2004).

The most accurate indicators of undifferentiating ability of iPS cells include cellular form, alkali phosphatase staining positive and retention of expression of NANOG. Cell differentiation can be evaluated by measuring the reduction in expression of NANOG. The expression of NANOG can be measured and evaluated by a known method such as PCR. The method is not limited to these.

The differentiation-induced hepatic progenitor cells and NANOG which is a marker for differentiation induction can be confirmed by measuring mRNA of a target protein in the cells after reverse transcription by a known method such as a polymerase chain reaction or real time-polymerase chain reaction (RT-PCR), or further by the ELISA method or immunostaining method using an antibody against the protein to be tested. The method is not limited to these methods.

The hepatic progenitor cells according to the present invention have higher proliferation capacity than that of mature hepatic cells, and form biliary epithelium as well, and thus, when transplanted into the liver, are considered to rapidly form the existing liver construction. The hepatic progenitor cells differentiation-induced from pluripotent stem cells according to the present invention can be administered to or transplanted into patients with hepatic failure.

Hepatic failure includes, but not limited to, acute hepatitis, chronic hepatitis, fulminant hepatitis, hepatic cirrhosis or liver cancer. Especially, fulminant hepatitis is accompanied by severe feeling since hospital admission and leads to hepatic failure within about 1 to 2 week(s). Since life saving is quite difficult once development of multiple organ failure, fulminant hepatitis must be treated, for example, by transplantation as soon as possible. At present, some medical institutions implement living donor liver transplantation. In this case, donors are generally family members. However, there are problems such as accompanying great invasion to donors, and the development of cell therapy, artificial organs and the like is demanded.

The pharmaceutical composition according to the present invention comprises a pharmacologically acceptable medicinal additive, in addition to the hepatic progenitor cell of the present invention. The pharmaceutical additives include, but not limited to, an isotonizing agent, a buffer, a pH adjuster, a stabilizer, a chelating agent, a bactericide and an antibacterial agent.

Examples of the isotonizing agent can include sodium chloride, potassium chloride, saccharides and glycerin. Examples of the buffer can include boric acid, phosphoric acid, acetic acid, citric acid and their corresponding salts (for example, alkali metal salts and alkali earth metal salts such as their sodium salt, potassium salt, calcium salt and magnesium salt). Examples of the pH adjuster can include: inorganic acids such as hydrochloric acid, sulfuric acid, phosphoric acid, polyphosphoric acid, boric acid or borax; organic acids such as acetic acid, propionic acid, oxalic acid, gluconic acid, fumaric acid, lactic acid, citric acid, succinic acid, tartaric acid and malic acid; inorganic bases such as potassium hydroxide or sodium hydroxide; organic bases such as monoethanolamine, triethanolamine, diisopropanolamine or triisopropanolamine; ammonium acetate, sodium lactate, sodium citrate, potassium carbonate, sodium hydrogen carbonate, sodium carbonate, ammonium hydrogen carbonate, dipottasium phosphate, potassium dihydrogen phosphate, sodium hydrogen phosphate, sodium dihydrogen phosphate and potassium lactate. Examples of the stabilizer can include human serum albumin, and normal L-amino acid, saccharides and cellulose derivatives, and these stabilizers can be used alone or in combination with a surfactant or the like. The L-amino acid is any of glycine, cysteine, glutamic acid and the like, but is not limited thereto. The saccharides are any of monosaccharides such as glucose, mannose, galactose and fructose; sugar alcohols such as mannitol, inositol and xylitol; disaccharides such as sucrose, maltose and lactose; polysaccharides such as dextran, hydroxypropyl starch, chondroitin sulfuric acid and hyaluronic acid; and their derivatives, but are not limited thereto. The cellulose derivatives are any of methylcellulose, ethylcellulose, hydroxyethylcellulose, hydroxypropylcellulose, hydroxypropylmethylcellulose and carboxymethylcellulose sodium, but are not limited thereto. Edetate sodium, citric acid and the like can be indicated as examples of the chelating agent.

As the medicinal additives such as isotonizing agent, pH adjuster, buffer, solubilizer and stabilizer, known compounds other than the medicinal additives exemplified above can be used in known dosage regimens and at known doses (for example, which are described in Dictionary of Medicinal Additives 2007 (edited by International Pharmaceutical Excipients Council Japan, YAKUJI NIPPO LIMITED, Tokyo, 2007)). However, the medicinal additives are not limited to these.

The hepatic progenitor cells differentiation-induced from pluripotent stem cells according to the present invention are cultured and maintained outside human bodies, and thus can also be used as an artificial liver.

According to the present invention, it is made possible to induce differentiation into hepatic progenitor cells in a culture dish in eight days by adhering human pluripotent stem cells to a substrate for monolayer culture without formation of embryoid bodies therefrom, accomplishing mass culture of hepatic progenitor cells in a short term.

### BRIEF DESCRIPTION OF THE DRAWINGS

FIG. 1 shows results of amplifying, by the PCR method, each mRNA of transcription factors which are observed to be expressed in fetal and adult livers, but not observed to be expressed in iPS cells and thereafter making an investigation by electrophoresis, in order to investigate the transcription factors. The respective lanes are Lane 1: water; Lane 2: iPS cells; Lane 3: fetal liver; and Lane 4: adult liver.

FIG. 2 shows results of investigating, by the PCR method and electrophoresis, the influences on the expression of transcription factors in iPS cells when the iPS cells were cultured in a medium to which various growth-promoting agents were added. The expression of SOX-17, GATA6, FOXA2, GATA4, HEX, TTR and C/EBPα as the transcription factors was investigated and evaluated. The growth-promoting agents in the respective lanes are Lane 1: water (blank); Lane 2: ReproFF medium; Lane 3: iPSm (−) medium; Lane 4: bFGF; Lane 5: BMP4; Lane 6: oncostatin M; Lane 7: EGF; Lane 8: NGF; Lane 9: TGF-β1; Lane 10: retinoic acid; and Lane 11: HGF.

FIG. 3-1 shows results of analysis on the induction of differentiation into hepatic progenitor cells by using a combination of growth-promoting agents. Analyzed were the influences of the growth-promoting agent combinations on the increase in expression of mRNA of α-fetoprotein which is an indicator protein for hepatic progenitor cells. As internal standard, RPL19 was measured also to obtain the α-fetoprotein/RPL19 ratio. The growth-promoting agents in the respective lanes are Lane 1: ReproFF medium; Lane 2: oncostatin M; Lane 3: epidermal growth factor; Lane 4: retinoic acid; Lane 5: dexamethasone; Lane 6: ITS; Lane 7: sample comprising oncostatin M, epidermal growth factor and retinoic acid added together; and Lane 8: sample comprising oncostatin M, epidermal growth factor, retinoic acid, dexamethasone and ITS added together.

FIG. 3-2 shows results of analysis on the induction of differentiation into hepatic progenitor cells by using a combination of growth-promoting agents. Analyzed were the influences of the growth-promoting agent combinations on the increase in expression of mRNA of DLK-1 which is an indicator protein for hepatic progenitor cells. As internal standard, RPL19 was measured also to obtain the DLK-1/RPL19 ratio. The growth-promoting agents in the respective lanes are Lane 1: ReproFF medium; Lane 2: oncostatin M; Lane 3: epidermal growth factor; Lane 4: retinoic acid; Lane 5: dexamethasone; Lane 6: ITS; Lane 7: sample comprising oncostatin M, epidermal growth factor and retinoic acid added together; and Lane 8: sample comprising oncostatin M, epidermal growth factor, retinoic acid, dexamethasone and ITS added together.

FIG. 4-1 shows results of obtaining the ratio of the amount of AFP expressed to the amount of RPL19 expressed and analyzing the induction of differentiation into hepatic progenitor cells by using a combination of various growth-promoting agents with respect to iPS cells into which the transcription factors, FOXA2, GATA4, HEX and C/EBPα were transfected. The numerical values for the respective experimental groups on the abscissa are 1: ReproFF medium; 2: GHA; 3: FHA; 4: FGA; 5: FGH; 6: FGHA and 7: fetal liver, wherein F represents FOXA2; G represents GATA4; H represents HEX; and A represents C/EBPα.

FIG. 4-2 shows results of obtaining the ratio of the amount of DLK-1 expressed to the amount of RPL19 expressed and analyzing the induction of differentiation into hepatic progenitor cells by using a combination of various growth-promoting agents with respect to iPS cells into which the transcription factors, FOXA2, GATA4, HEX and C/EBPα were transfected. The numerical values for the respective experimental groups on the abscissa are 1: ReproFF medium; 2: GHA; 3: FHA; 4: FGA; 5: FGH; 6: FGHA and 7: fetal liver, wherein F represents FOXA2; G represents GATA4; H represents HEX; and A represents C/EBPα.

FIG. 4-3 shows results of obtaining the ratio of the amount of G-GTP expressed to the amount of RPL19 expressed and analyzing the induction of differentiation into hepatic progenitor cells by using a combination of various growth-promoting agents with respect to iPS cells into which the transcription factors, FOXA2, GATA4, HEX and C/EBPα were transfected. The numerical values for the respective experimental groups on the abscissa are 1: ReproFF medium; 2: GHA; 3: FHA; 4: FGA; 5: FGH; 6: FGHA and 7: fetal liver, wherein F represents FOXA2; G represents GATA4; H represents HEX; and A represents C/EBPα.

FIG. 4-4 shows results of obtaining the ratio of the amount of NANOG expressed to the amount of RPL19 expressed and analyzing the induction of differentiation into hepatic progenitor cells by using a combination of various growth-promoting agents with respect to iPS cells into which the transcription factors, FOXA2, GATA4, HEX and C/EBPα were transfected. The numerical values for the respective experimental groups on the abscissa are 1: ReproFF medium; 2: GHA; 3: FHA; 4: FGA; 5: FGH; 6: FGHA and 7: fetal liver, wherein F represents FOXA2; G represents GATA4; H represents HEX; and A represents C/EBPα.

FIG. 5-1 shows a micrograph for investigation on indocyanine green (ICG) incorporation of hepatic progenitor cells differentiation-induced from induced pluripotent stem cells according to the present invention. This figure is a drawing figure from a phase-contrast micrograph of culture of hepatic progenitor cells differentiation-induced from undifferentiated human iPS cells by a method according to the present invention. The photograph shown in FIG. 5-1 was taken at 200-fold magnification, and the scale bar indicates 25 μm.

FIG. 5-2 shows a micrograph for investigation on indocyanine green incorporation of hepatic progenitor cells differentiation-induced from induced pluripotent stem cells according to the present invention. This figure is a drawing figure from a phase-contrast micrograph of culture involving addition of indocyanine green to hepatic progenitor cells differentiation-induced from undifferentiated human iPS cells by a method according to the present invention. The photograph shown in FIG. 5-2 was taken at 200-fold magnification, and the scale bar indicates 25 μm.

FIG. 5-3 shows a micrograph for investigation on indocyanine green incorporation of hepatic progenitor cells differentiation-induced from induced pluripotent stem cells according to the present invention. This figure is a drawing figure from a photograph in which green portions (green is a fluorescent color of indocyanine green) in the original figure (color) in FIG. 5-1 were converted and emphasized as white portions. The arrows indicate green portions in the original figure. The photograph in FIG. 5-3 was taken at 200-fold magnification, and the scale bar indicates 25 μm.

FIG. 5-4 shows a micrograph for investigation on indocyanine green (ICG) incorporation of hepatic progenitor cells differentiation-induced from induced pluripotent stem cells according to the present invention. This figure is a drawing figure from a photograph in which green portions (green is a fluorescent color of indocyanine green) in the original figure (color) in FIG. 5-2 was converted and emphasized as white portions. The arrows indicate green portions in the original figure. The photograph in FIG. 5-4 was taken at 200-fold magnification, and the scale bar indicates 25 μm.

### MODES FOR CARRYING OUT THE INVENTION

Examples of the present invention which will be explained below are given only for illustrative purposes, and are not intended to limit the technical scope of the present invention. The technical scope of the present invention is limited only by the claims. Modifications of the present invention, including addition, deletion and substitution of the essential features of the present invention, can be made without departing the purport of the present invention.

### Example 1

### Search of Transcription Factors which are Expressed in Fetal and Adult Hepatic Cells and are Unexpressed in iPS Cells

### 1.1 Experimental Materials and Methods

Reverse transcriptase (Life Technologies Japan Ltd.) was used to synthesize cDNAs of iPS cells, fetal hepatic cells and adult hepatic cells from 3 μg of RNA. The cDNAs were subjected to polymerase chain reaction (PCR) using the following primers to GATA4, FOXA2, HEX, C/EBPα and C/EBPβ, respectively, and to electrophoresis using 2% low-melting agarose (Lonza) and 1×TAE, for investigations on the expression of GATA4, FOXA2, HEX, C/EBPα and C/EBNβ in the iPS cells, fetal hepatic cells and adult hepatic cells.

After electrophoresis, the 2% low-melting agarose was irradiated with ultraviolet rays at 254 nm from a UV transilluminator (UVP LLC, NLMS-20E), and a Polaroid photograph (FUJIFILM Corporation, FP-3000B) thereof was taken with a gel camera (Funakoshi DS-300) to analyze an electrophoretic pattern.

PCR cycles performed included 30 cycles of thermal denaturation: 1 minute at 94° C.; annealing: 1 minute and elongation reaction: 1 minute at 72° C.

Primer nucleotide sequence of GATA4:
Forward;
(SEQ ID NO: 1)
5′-GAAAACGGAAGCCCAAGAACC-3′
Reverse;
(SEQ ID NO: 2)
5′-AGACATCGCACTGACTGAGAACG-3′

Annealing was performed at a temperature of 55.9° C.

Primer nucleotide sequence of FOXA2:
Forward;
(SEQ ID NO: 3)
5′-CCACCACCAACCCCACAAAATG-3′
Reverse;
(SEQ ID NO: 4)
5′-TGCAACACCGTCTCCCCAAAGT-3′

Annealing was performed at a temperature of 60° C.

Primer nucleotide sequence of HEX:
Forward;
(SEQ ID NO: 5)
5′-TTCTCCAACGACCAGACCATCG-3′
Reverse;
(SEQ ID NO: 6)
5′-TTTTATCGCCCTCAATGTCCAC-3′

Annealing was performed at a temperature of 56.2° C.

Primer nucleotide sequence of C/EBPα:
Forward;
(SEQ ID NO: 7)
5′-TGGAGACGCAGCAGAAGGTG-3′
Reverse;
(SEQ ID NO: 8)
5′-TCGGGAAGGAGGCAGGAAAC-3′

Annealing was performed at a temperature of 69.1° C.

Primer nucleotide sequence of C/EBPβ:
Forward;
(SEQ ID NO: 9)
5′-CCAAGAAGACCGTGGACAAGC-3′
Reverse;
(SEQ ID NO: 10)
5′-AAGTTCCGCAGGGTGCTGAG-3′

Annealing was performed at a temperature of 59.5° C.

### 1.2 Experimental Results

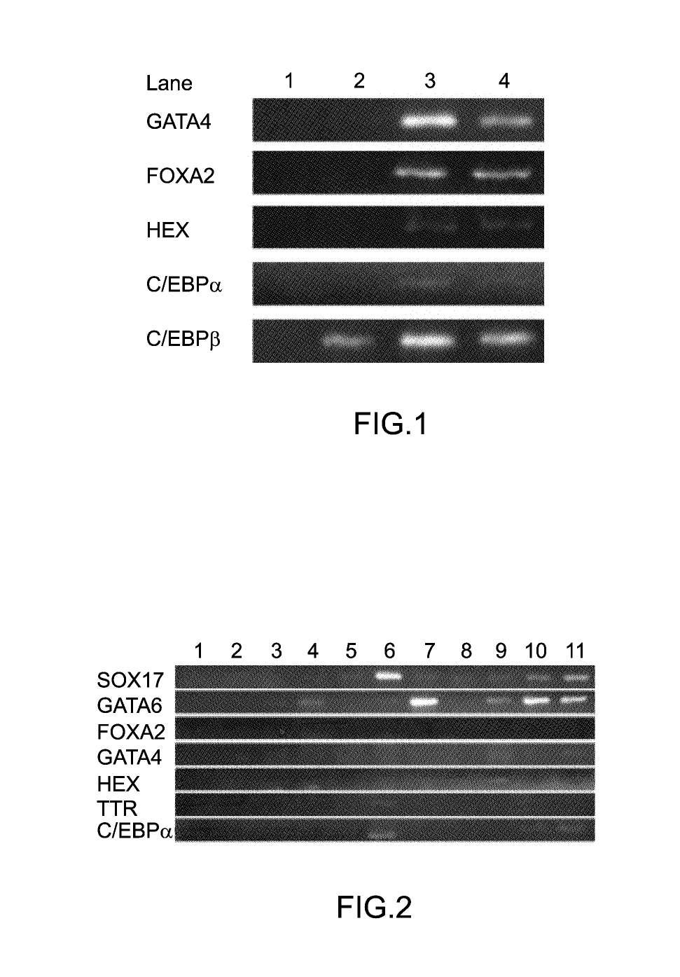

The results of electrophoresis are shown in FIG. 1. The respective lanes are Lane 1: water; Lane 2: iPS cells; Lane 3: fetal liver; and Lane 4: adult liver.

In this experiment, GATA4, FOXA2, HEX and C/EBPα were observed to be expressed in fetal and adult livers, but not observed to be expressed in iPS cells. C/EBPβ was observed to be expressed both in iPS cells and in fetal and adult livers.

### Example 2

### Investigations on Transcription Factors which are Unexpressed by Using Growth-Promoting Agents Alone

### 2.1 Experimental Materials and Methods

Human iPS cells (201B7, RIKEN cell bank) were seeded onto a matrigel-coated 6-well plate, and cultured in a feeder-less medium, ReproFF (trade name, Reprocell Inc.) for culture of stem cells maintained in an undifferentiated state, without using a feeder cell, under conventional conditions: 37° C. and 5% carbon dioxide.

A medium obtained by adding a 20% knock-out serum replacement (Life Technologies Japan Ltd.), 10% Minimum Essential Amino Acids (Life Technologies Japan Ltd.), 2 mM of L-glutamine (Life Technologies Japan Ltd.) and 1 mM of 2-mercaptoethanol to a D-MEM/F12 medium (Dulbecco's Modified Eagle Medium-F12 medium, Sigma-Aldrich Japan) was used as an iPSm(−) medium.

The following growth-promoting agents were added to the iPSm(−) medium, and SOX-17, GATA6, FOXA2, GATA4, HEX, TTR and C/EBPα were expressed in a manner similar to that in Experiment 1.

As the growth-promoting agents, added were bFGF (basic fibroblast growth factor, Wako Pure Chemical Industries, Ltd.), BMP (bone morphogenetic protein)-4 (Wako Pure Chemical Industries, Ltd.), oncostatinM (Wako Pure Chemical Industries, Ltd.), an epidermal growth factor (EGF, Wako Pure Chemical Industries, Ltd.), a nerve growth factor (NGF, R & D Systems, Inc.), TGF-β, (transform growth factor-β, R& D Systems, Inc.), retinoic acid (Sigma-Aldrich Japan) and a hepatic cell growth factor (HGF, Sigma-Aldrich Japan).

RT-PCR to SOX-17, GATA6 and TTR was performed under the following conditions.

Primer nucleotide sequence of SOX-17:
Forward;
(SEQ ID NO: 11)
5′-CGCTTTCATGGTGTGGGCTAAGGACG-3′
Reverse;
(SEQ ID NO: 12)
5′-TAGTTGGGGTGGTCCTGCATGTGCTG-3′

Annealing was performed at a temperature of 63° C.

Primer nucleotide sequence of GATA6:
Forward;
(SEQ ID NO: 13)
5′-TTCATCACGGCGGCTTGGATTGTC-3′
Reverse;
(SEQ ID NO: 14)
5′-GTGTTGTGGGGGAAGTATTTTTGC-3′

Annealing was performed at a temperature of 55.9° C.

Primer nucleotide sequence of TTR:
Forward;
(SEQ ID NO: 15)
5′-GGTGAATCCAAGTGTCCTCTGAT-3′
Reverse;
(SEQ ID NO: 16)
5′-GTGACGACAGCCGTGGTGGAA-3′

Annealing was performed at a temperature of 61° C.

### 2.2 Experimental Results

The results of electrophoresis are shown in FIG. 2. The respective lanes are Lane 1: water; Lane 2: ReproFF; Lane 3: iPSm(−); and Lane 4: bFGF; Lane 5: BMP-4; Lane 6: oncostatin M; Lane 7: EGF; Lane 8: NGF; Lane 9: TGF-β1; Lane 10: retinoic acid; and Lane 11: HGF.

SOX-17 was observed to be expressed by oncostatin M, and GATA6 was observed to be expressed by EGF, TGF-β and retinoic acid. C/EBPα was observed to be slightly expressed by oncostatin M. On the other hand, FOXA2, GATA4, HEX and C/EBPα were not observed to be expressed by these growth-promoting agents (oncostatin M, EGF, TGF-β and retinoic acid).

### Example 3

### Analysis on Induction of Differentiation into Hepatic Progenitor Cells by Using a Combination of Growth-Promoting Agents

Based on the results of Example 2, for the transcription factors, FOXA2, GATA4, HEX and C/EBPα which were not observed to be expressed upon addition of growth-promoting agents, the respective expression vectors thereof were introduced into human induced pluripotent stem cell to attempt to induce differentiation into hepatic progenitor cells.

### 3.1 Experimental Materials and Methods

Human iPS cells (201B7, RIKEN cell bank) were seeded onto a matrigel-coated 6-well plate, and cultured in a medium, ReproFF in accordance with a conventional method under the following conditions: 37° C. and 5% carbon dioxide. A lipofection reagent, Lipofectamine LTX (registered trade mark, Life Technologies Japan Ltd.) was used to transfect 0.5 μg of expression plasmids of FOXA2, GATA4, HEX and C/EBPα, respectively.

As the expression vectors of the transcription factors, used were expression vectors (human TrueClone, OriGene Technologies, Inc., COSMO BIO co., ltd.) in which full-length cDNAs of human FOXA2, GATA4, HEX and CEBPA were each incorporated in the downstream of a potent-promoting agent derived from cytomegalovirus. In the human FOXA2 expression vector (catalog No. sc122913), the full-length cDNA coding human FoxA2 protein was inserted between EcoR1 and Sal1 restricted sites of pCMV6-XL5. In the human GATA4 expression vector (catalog No. sc124037), the full-length cDNA coding human GATA4 protein was inserted between EcoR1 and Sal1 restricted sites of pCMV6-XL4. In the human HEX expression vector (catalog No. sc321626), the full-length cDNA coding human HHEX protein was inserted between Sgf1 and Mlu1 restricted sites of pCMV6-AC. In the human CEBPA expression vector (catalog No. sc303472), the full-length cDNA coding human CEBPA protein was inserted between EcoR1 and Sal1 restricted sites of pCMV6-XL5.

Immediately before transfection, the medium to be used was changed to a medium obtained by adding oncostatin M (Wako Pure Chemical Industries, Ltd.), EGF (Wako Pure Chemical Industries, Ltd.) and retinoic acid (Wako Pure Chemical Industries, Ltd.) to the iPSm(−) medium. Here, the iPSm(−) medium corresponds to a medium obtained by eliminating a basic fibroblast growth factor from a medium for feeder cells of human iPS cells recommended by CiRA at Kyoto University. Specifically, a medium obtained by adding a 20% knock-out serum replacement (KSR, Life Technologies Japan Ltd.), 10% Minimum Essential Amino Acids (Life Technologies Japan Ltd.), 2 mM of L-glutamine (Life Technologies Japan Ltd.) and 0.1 mM of 2-mercaptoethanol to a D-MEM/F12 medium (Dulbecco's Modified Eagle Medium-F12 medium, Sigma-Aldrich Japan) was used as the an iPSm(−) medium.

The transfection of the transcription factors was repeated three times every three days, and, on the 8th day, RNA was extracted using Isogen (NIPPON GENE CO., LTD.). From this RNA, cDNA was synthesized using a superscript III first strand system (Life Technologies Japan Ltd.). The cDNA was diluted twentyfold. Then, the amount of expressed α-fetoprotein (AFP), which was an indicator of hepatic progenitor cells, was analyzed by the real-time quantitative PCR method using a real-time PCR analysis reagent, Fast SYBR Green Master Mix (trade name, Life Technologies Japan Ltd.). In the meantime, a real-time PCR detection apparatus, MiniOpticon (Bio-Rad) was used for analysis. The amounts of α-fetoprotein (AFP) and Delta-like (DLK)-1 expressed were quantitatively determined as an indicator of hepatic progenitor cells by employing ribosome-related protein (RLP19) as internal standard (each group, n=3).

The real-time quantitative PCR was performed by using the following primers through a PCR cycle including 30 cycles of 5 seconds at 95° C. and 20 seconds at 60° C.

Primer nucleotide sequence of AFP: (147 bp)
Forward;
(SEQ ID NO: 17)
5′-ACACAAAAAGCCCACTCCAG-3′
Reverse;
(SEQ ID NO: 18)
5′-GGTGCATACAGGAAGGGATG-3′

Primer nucleotide sequence of DLK-1: (121 bp)
Forward;
(SEQ ID NO: 19)
5′-GGATGAGTGCGTCATAGCAA-3′
Reverse;
(SEQ ID NO: 20)
5′-CCTCCTCTTCAGCAGCATTC-3′

Primer nucleotide sequence of RLP19: (157 bp)
Forward;
(SEQ ID NO: 21)
5′-CGAATGCCAGAGAAGGTCAC-3′
Reverse;
(SEQ ID NO: 22)
5′-CCATGAGAATCCGCTTGTTT-3′

### 3.2 Experimental Results

The results of investigations on increase in expression of α-fetoprotein due to addition of the respective growth-promoting agents are shown in FIGS. 3-1 and 3-2. The numerical values on the abscissa in the bar graphs shown in FIGS. 3-1 and 3-2 indicate those when the following growth-promoting agents were added to the medium. The error bar indicates a standard error. The respective numbers are 1: ReproFF, 2: oncostatin M, 3: epidermal growth factor, 4: retinoic acid; 5: dexamethasone; 6: ITS; 7: oncostatin M, epidermal growth factor and retinoic acid added together; 8: oncostatin M, epidermal growth factor, retinoic acid, dexamethasone and (insulin, transferrin and selenite ion hereinafter collectively abbreviated as “ITS”) added together.

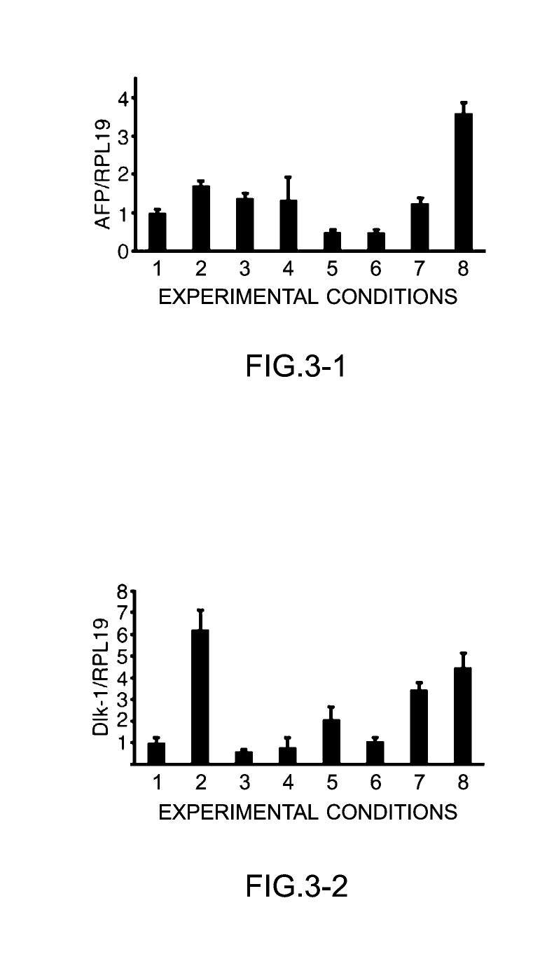

As shown in FIG. 3-1, the expression of α-fetoprotein was most increased in the group cultured in a medium comprising oncostatin M, epidermal growth factor, retinoic acid, dexamethasone and ITS added together, and the second best group was the group with addition of oncostatin M.

On the other hand, as shown in FIG. 3-2, the expression of DLK-1 was most increased in the group with addition of oncostatin M, and the second best group was the group with co-addition of oncostatin M, epidermal growth factor, retinoic acid, dexamethasone and ITS.

The numerical values for the respective lanes shown in FIG. 3-1 are Lane 1: 100±11; Lane 2: 172±11; Lane 3: 139±13; Lane 4: 133±58; Lane 5: 50.1±5; Lane 6: 49±7; Lane 7: 125±13; and Lane 8: 359±26. The numerical values for the respective lanes shown in FIG. 3-2 are Lane 1: 100±25; Lane 2: 623±86; Lane 3: 59±7; Lane 4: 79±40; Lane 5: 208±54; Lane 6: 106±19; Lane 7: 346±31; and Lane 8: 449±66.

From the above results, it has been indicated that the group with co-addition of oncostatin M, epidermal growth factor, retinoic acid, dexamethasone and ITS is most effective for increase in expression of both α-fetoprotein and DLK-1.

### Example 4

### Analysis on Induction of Differentiation into Hepatic Progenitor Cells by Using a Combination of Transcription Factors

### 4.1 Experimental Materials and Methods

The transcription factors FOXA2 (hereinafter abbreviated as F), GATA4 (hereinafter abbreviated as G), HEX (hereinafter abbreviated as H) and C/EBPα (hereinafter abbreviated as A) were transfected three times using Lipofectamine LTX every three days. As growth-promoting agents, EGF, retinoic acid, oncostatin M, dexamethasone and ITS ware added. On the 8th day, real-time quantitative RT-PCR was performed in a manner similar to that employed in Example 3.

AFP and DLK-1 were analyzed as indicators of hepatic progenitor cells, and G-GTP was analyzed as an indicator of the ability to differentiate into biliary epithelial cells. Further, NANOG was analyzed as an indicator of the undifferentiation potency.

The real-time quantitative RT-PCR was performed under the following conditions through a PCR cycle including 30 cycles of 5 seconds at 95° C. and 20 seconds at 60° C.

Primer nucleotide sequence of G-GTP:
Forward;
(SEQ ID NO: 23)
5′-CCTCATCCTCAACATCCTCAAAGG-3′
Reverse;
(SEQ ID NO: 24)
5′-CACCTCAGTCACATCCACAAACTTG-3′

Primer nucleotide sequence of NANOG:
Forward;
(SEQ ID NO: 25)
5′-CCGTTTTTGGCTCTGTTTTG-3′
Reverse;
(SEQ ID NO: 26)
5′-TCATCGAAACACTCGGTGAA-3′

### 4.2 Experimental Results

The results are shown in FIGS. 4-1, 4-2, 4-3 and 4-4. The respective lanes are Lane 1: ReproFF; Lane 2: combination of GHC (GATA4, HEX and C/EBPα); Lane 3: combination of FHA (FOXA2, HEX and C/EBPα); Lane 4: combination of FGA (FOXA2, GATA4 and C/EBPα); Lane 5: combination of FGH (FOXA2, GATA4 and HEX); Lane 6: combination of FGHA (FOXA2, GATA4, HEX and C/EBPα); and Lane 7: fetal liver.

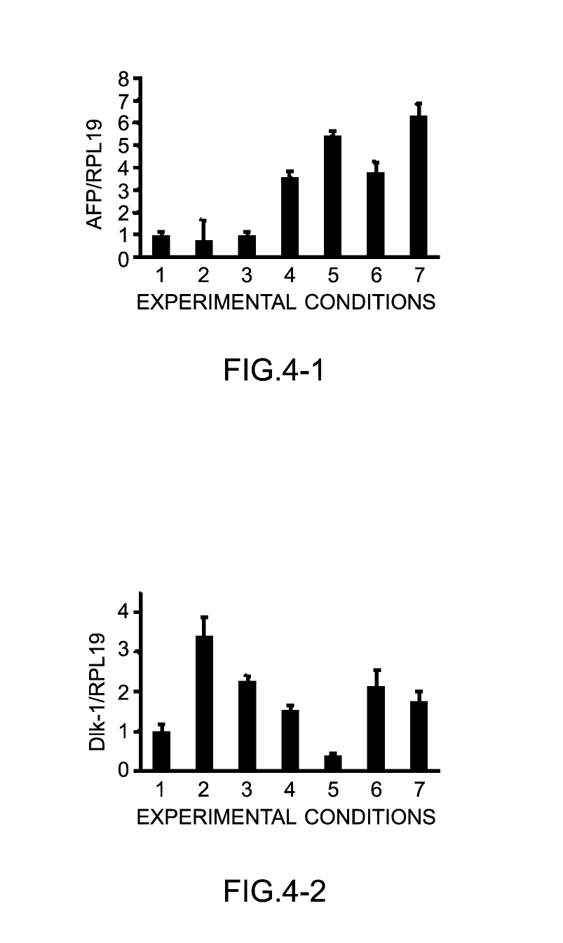

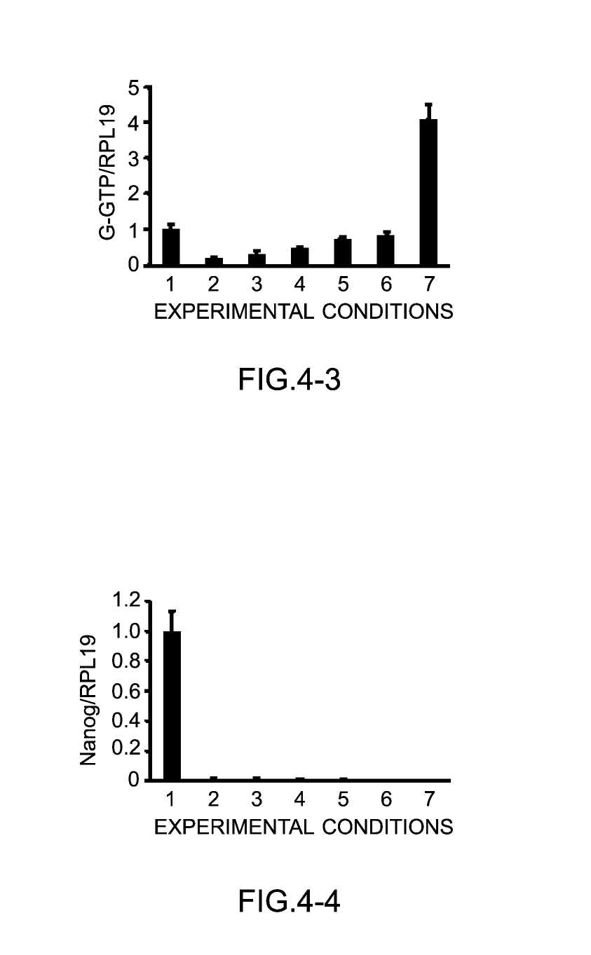

In the experimental group into which four kinds of expression vectors of FOXA2, GATA4, HEX and C/EBPα were simultaneously transfected, AFP (FIG. 4-1), DLK-1 (FIG. 4-2) and G-GTP (FIG. 4-3) were expressed, and the expression of NANOG (FIG. 4-4) was reduced. Especially, the result was obtained that, in the lane into which four kinds of expression vectors of FOXA2, GATA4, HEX and C/EBPα were transfected, G-GTP was most potently expressed as compared with the transfection of three kinds of expression vectors among these.

From these results, it has been revealed that the combination of the transcription factors FOXA2, GATA4, HEX and C/EBPα and the combination of the growth-promoting agents EGF, retinoic acid, oncostatin M, dexamethasone and ITS most efficiently induce the differentiation of iPS cells into hepatic progenitor cells.

The numerical values for the respective lanes shown in FIG. 4-1 are Lane 1: 100±18; Lane 2: 75±93; Lane 3: 98±13; Lane 4: 359±29; Lane 5: 544±20; Lane 6: 378±45; and Lane 7: 629±54. The numerical values for the respective lanes shown in FIG. 4-2 are Lane 1: 100±17; Lane 2: 339±48; Lane 3: 226±14; Lane 4: 153±10; Lane 5: 39±3; lane 6: 215±41; and Lane 7: 175±22. The numerical values for the respective lanes shown in FIG. 4-3 are Lane 1: 100±15; Lane 2: 20±3; Lane 3: 31±6; Lane 4: 47±6; Lane 5: 75±7; Lane 6: 83±4; and Lane 7: 408±36. The numerical values for the respective lanes shown in FIG. 4-4 are Lane 1: 100±14; Lane 2: 0.55±0.3; Lane 3: 1.16±0.4; Lane 4: 1.0±0.2; Lane 5: 1.2±0.4; Lane 6: 0.45±0.05; and Lane 7: 0.17±0.05.

### Example 5

### Analysis on Indocyanine Green (ICG) Incorporation Function of Cells Differentiation-Induced into Hepatic Progenitor Cells

### 5.1 Experimental Materials and Methods

Based on the results of Examples 3 and 4, the transfection of FOXA2, GATA4, HEX and C/EBPα into induced pluripotent stem cells was repeated three times using Lipofectamine LTX every three days in a similar manner, and these cells were cultured in a medium to which EGF, retinoic acid, oncostatin M, dexamethasone and ITS were added as growth-promoting agents. As for ICG actively incorporated only by hepatic cells in adult, ICG (Santen Pharmaceutical Co., Ltd.) was added at a concentration of 1 mg/mL on the 8th day of differentiation induction based on Example 3. Fifteen (15) minutes after the addition, the incorporation of ICG into the cells was observed with an optical microscope, CKX41N-31PHP (OLYMPUS CORPORATION).

### 5.2 Experimental Results

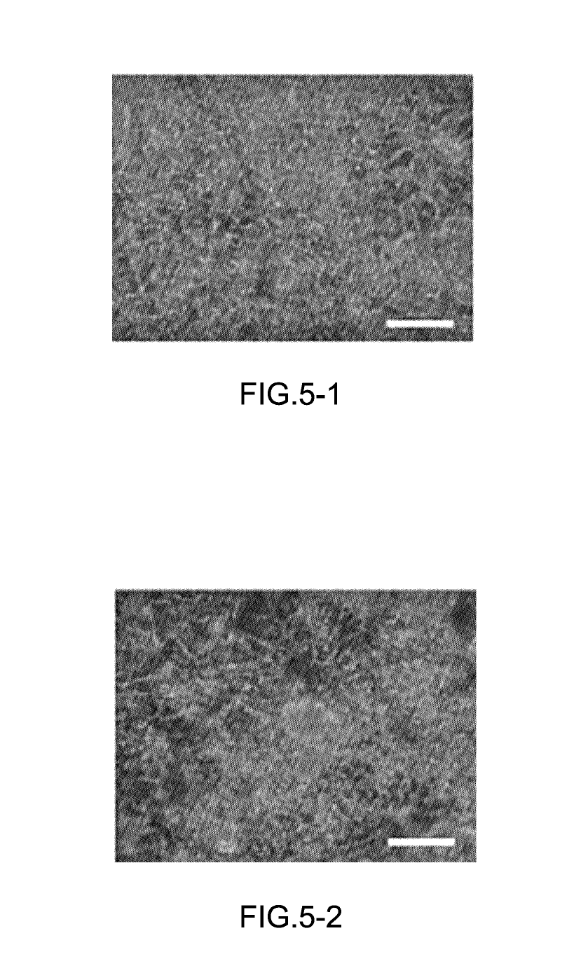

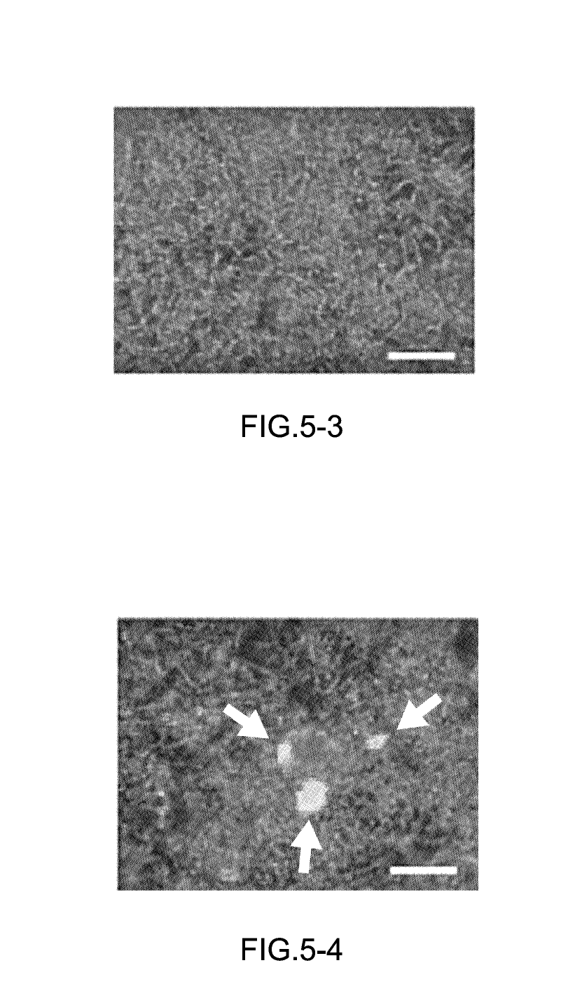

FIGS. 5-1 and 5-2 show phase-contrast micrographs (×200) of 201B7 cells on the 8th day in which the four kinds of transcription factors were subjected to lipofection three times in the presence of the five kinds of differentiation-inducing agents. The scale bar indicates 25 μm. FIG. 5-1 shows a photograph of cells not subjected to indocyanine green treatment, and FIG. 5-2 shows a photograph of cells subjected to indocyanine green treatment. FIGS. 5-3 and 5-4 show photographs in which green portions in the original figures (color) in FIGS. 5-1 and 5-2 were converted and emphasized as white portions. The arrows in FIG. 5-4 indicate green portions in the original figures. As shown in this figure, cells into which indocyanine green was incorporated and thus stained green were observed in the culture of 201B7 cells on the 8th day in which the four kinds of transcription factors were subjected to lipofection three times in the presence of the five kinds of differentiation-inducing agents. Indocyanine green is known to be incorporated only into hepatic cells from the blood circulation. It has been indicated that the hepatic progenitor cells differentiation-induced from undifferentiated human iPS cells by the method of the present invention in eight days exhibit the function of hepatic cells.

1. A method of inducing the differentiation of an undifferentiated human pluripotent stem cell to obtain a hepatic progenitor cell, comprising a step of expressing a combination of transcription factors for differentiation induction in the human pluripotent stem cell which is adhered to a substrate for monolayer culture in a medium for differentiation.

2. The method according to claim 1, wherein the transcription factors for differentiation induction are FOXA2, GATA4, HEX and C/EBPα.

3. The method according to claim 1, wherein the medium for differentiation further comprises a combination of growth-promoting agents.

4. The method according to claim 3, wherein the combination of growth-promoting agents includes one or more of the growth-promoting agents selected from the group consisting of oncostatin M, an epidermal growth factor, retinoic acid, dexamethasone, insulin, transferrin and a selenite ion.

5. The method according to claim 1, wherein, in the step of expressing the combination of transcription factors for differentiation induction according to claim 1 in the human pluripotent stem cell, the combination of transcription factors for differentiation induction is transiently expressed in the human pluripotent stem cell in a repetitive manner.

6. The method according to claims 1, wherein the human pluripotent stem cell is a human induced pluripotent stem cell.

7. A cell composition for transplantation into the liver, comprising a human pluripotent stem cell-derived hepatic progenitor cell obtained by the method according to claim 1.

8. A method of inducing the differentiation of human induced pluripotent stem cell (human iPS cell) to obtain a human hepatic progenitor cell, comprising steps of:
transfecting genes of transcription factors FOXA2, GATA4, HEX and C/EBPα, respectively, into a human iPS cell every three days;
carrying out differentiation induction in a medium containing oncostatin M, an epidermal growth factor, retinoic acid, dexamethasone, insulin, transferrin and a selenite ion as a combination of growth-promoting agents; and
obtaining a human hepatic progenitor cell by inducing the differentiation of the human iPS cell, on the 8th day after the transfection.

9. A human hepatic progenitor cell obtained by inducing the differentiation of a human induced pluripotent stem cell (human iPS cell), wherein
genes of transcription factors FOXA2, GATA4, HEX and C/EBPα, respectively, are transfected into a human iPS cell every three days, and
differentiation induction is carried out in a medium containing oncostatin M, an epidermal growth factor, retinoic acid, dexamethasone, insulin and transferrin as a combination of growth-promoting agents to obtain a human hepatic progenitor cell on the 8th day after the transfection.

10. A cell composition for transplantation into the liver, comprising the human hepatic progenitor cell according to claim 9.


SEQUENCE LISTING (30 sequences)

SEQ ID NO: 1 - DNA, 21 bp, Artificial
  GATA4 forward primer
gaaaacggaagcccaagaacc

SEQ ID NO: 2 - DNA, 23 bp, Artificial
  GATA4 reverse primer
agacatcgcactgactgagaacg

SEQ ID NO: 3 - DNA, 22 bp, Artificial
  FOXA2 forward primer
ccaccaccaaccccacaaaatg

SEQ ID NO: 4 - DNA, 22 bp, Artificial
  FOXA2 reverse primer
tgcaacaccgtctccccaaagt

SEQ ID NO: 5 - DNA, 22 bp, Artificial
  HHEX forward primer
ttctccaacgaccagaccatcg

SEQ ID NO: 6 - DNA, 22 bp, Artificial
  HHEX reverse primer
ttttatcgccctcaatgtccac

SEQ ID NO: 7 - DNA, 20 bp, Artificial
  CEBPA forward primer
tggagacgcagcagaaggtg

SEQ ID NO: 8 - DNA, 20 bp, Artificial
  CEBPA reverse primer
tcgggaaggaggcaggaaac

SEQ ID NO: 9 - DNA, 21 bp, Artificial
  CEBPB forward primer
ccaagaagaccgtggacaagc

SEQ ID NO: 10 - DNA, 20 bp, Artificial
  CEBPB reverse primer
aagttccgcagggtgctgag

SEQ ID NO: 11 - DNA, 26 bp, Artificial
  SOX17 forward primer
cgctttcatggtgtgggctaaggacg

SEQ ID NO: 12 - DNA, 26 bp, Artificial
  CEBPB reverse primer
tagttggggtggtcctgcatgtgctg

SEQ ID NO: 13 - DNA, 24 bp, Artificial
  GATA6 forward primer
ttcatcacggcggcttggattgtc

SEQ ID NO: 14 - DNA, 24 bp, Artificial
  GATA6 reverse primer
gtgttgtgggggaagtatttttgc

SEQ ID NO: 15 - DNA, 23 bp, Artificial
  TTR forward primer
ggtgaatccaagtgtcctctgat

SEQ ID NO: 16 - DNA, 21 bp, Artificial
  TTR reverse primer
gtgacgacagccgtggtggaa

SEQ ID NO: 17 - DNA, 20 bp, Artificial
  AFP forward primer
acacaaaaagcccactccag

SEQ ID NO: 18 - DNA, 20 bp, Artificial
  AFP reverse primer
ggtgcatacaggaagggatg

SEQ ID NO: 19 - DNA, 20 bp, Artificial
  DLL1 forward primer
ggatgagtgcgtcatagcaa

SEQ ID NO: 20 - DNA, 20 bp, Artificial
  DLL1 reverse primer
cctcctcttcagcagcattc

SEQ ID NO: 21 - DNA, 20 bp, Artificial
  RPL19 forward primer
cgaatgccagagaaggtcac

SEQ ID NO: 22 - DNA, 20 bp, Artificial
  RPL19 reverse primer
ccatgagaatccgcttgttt

SEQ ID NO: 23 - DNA, 24 bp, Artificial
  GGT1 forward primer
cctcatcctcaacatcctcaaagg

SEQ ID NO: 24 - DNA, 25 bp, Artificial
  GGT1 reverse primer
cacctcagtcacatccacaaacttg

SEQ ID NO: 25 - DNA, 20 bp, Artificial
  NANOG forward primer
ccgtttttggctctgttttg

SEQ ID NO: 26 - DNA, 20 bp, Artificial
  NANOG reverse primer
tcatcgaaacactcggtgaa

SEQ ID NO: 27 - protein, 463 aa, Homo sapiens
  MISC_FEATURE: FOXA2 protein (NP_068556.2)
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 28 - protein, 442 aa, Homo sapiens
  MISC_FEATURE: GATA4 protein (NP_002043.2)
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 29 - protein, 270 aa, Homo sapiens
  MISC_FEATURE: HHEX protein (NP_002720.1)
MQYPHPGPAAGAVGVPLYAPTPLLQPAHPTPFYIEDILGRGPAAPTPAPTLPSPNSSFTSLVSPYRTPVYEPTPIHPAFSHHSAAALAAAYGPGGFGGPLYPFPRTVNDYTHALLRHDPLGKPLLWSPFLQRPLHKRKGGQVRFSNDQTIELEKKFETQKYLSPPERKRLAKMLQLSERQVKTWFQNRRAKWRRLKQENPQSNKKEELESLDSSCDQRQDLPSEQNKGASLDSSQCSPSPASQEDLESEISEDSDQEVDIEGDKSYFNAG

SEQ ID NO: 30 - protein, 358 aa, Homo sapiens
  MISC_FEATURE: CEBPA protein (NP_004355.2)
MESADFYEAEPRPPMSSHLQSPPHAPSSAAFGFPRGAGPAQPPAPPAAPEPLGGICEHETSIDISAYIDPAAFNDEFLADLFQHSRQQEKAKAAVGPTGGGGGGDFDYPGAPAGPGGAVMPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA